(19) United States
(12) Patent Application Publication
(10) Pub. No.: US 2022/0406473 A1
(43) Pub. Date: Dec. 22, 2022

(54) REMOTE VIRTUAL AND AUGMENTED REALITY MONITORING AND CONTROL SYSTEMS

(71) Applicant: XR Health IL LTD, Tel Aviv (IL)

(72) Inventors: Tal Arbel, Hod-Hasharon (IL); Sagie Grunhaus, Tel Aviv (IL); Miki Levy, Misgav Dov (IL); Eran Orr, Brookline, MA (US)

(21) Appl. No.: 17/590,461

(22) Filed: Feb. 1, 2022

Related U.S. Application Data
(63) Continuation of application No. PCT/US20/44760, filed on Aug. 3, 2020.
(60) Provisional application No. 62/882,122, filed on Aug. 2, 2019.

(51) Int. Cl. G16H 80/00; G06F 3/01; G16H 20/30

(52) U.S. Cl. CPC G16H 80/00; G06F 3/011; G16H 20/30; G06V 40/15

(57) ABSTRACT

Provided herein are control and monitoring protocols for a virtual or augmented reality environment provided to patients, and which enable a trainer to have real-time monitoring and control of the virtual reality environment. In various embodiments, a virtual environment is provided to a first user at a first location. Data (e.g., biometric data) is collected based on the first user's interaction with the virtual environment while engaging in a training protocol. A real-time mirrored view of the virtual environment is provided to a second user via a network. The data and one or more control tools are provided to the second user. The control tools are configured to adjust one or more parameters of the virtual environment. A selection of at least one of the control tools is received form the second user and the virtual environment is adjusted based on the selection.

### CROSS-REFERENCE TO RELATED APPLICATION

This application is a continuation of International Application No. PCT/US2020/044760, filed Aug. 3, 2020, which claims the benefit of U.S. Provisional Patent Application No. 62/882,122, filed on Aug. 2, 2019, each of which is hereby incorporated by reference in its entirety.

### BACKGROUND

Embodiments of the present disclosure relate to remote virtual and augmented reality monitoring and control systems.

### BRIEF SUMMARY

According to embodiments of the present disclosure, systems for, methods of, and computer program products for control and monitoring of a virtual or augmented reality environment provided to patients are provided. In various embodiments, a virtual environment is provided to a first user via a virtual or augmented reality system at a first location. A first set of data is collected based on interaction of a first user with the virtual environment. The first set of data includes biometric data of the first user as the user engages in a training protocol received from a database. A real-time mirrored view of the virtual environment provided to the first user is provided to a second user via a network. The first set of data is provided to the second user via the network. One or more control tools is provided to the second user. The one or more control tools is configured to adjust one or more parameters of the virtual environment. A selection of at least one of the control tools is received from the second user. The virtual environment is adjusted based on the user selection.

In various embodiments, a system is provided including a virtual or augmented reality system, comprising a virtual or augmented reality display adapted to display a virtual environment to a first user, one or more biometric sensors coupled to the first user, and a computing node comprising a computer readable storage medium having program instructions embodied therewith. The program instructions are executable by a processor of the computing node to cause the processor to perform a method where a virtual environment is provided to a first user via a virtual or augmented reality system at a first location. A first set of data is collected from the one or more biometric sensors based on interaction of a first user with the virtual environment. The first set of data includes biometric data of the first user as the user engages in a training protocol received from a database. A real-time mirrored view of the virtual environment provided to the first user is provided to a second user via a network. The first set of data is provided to the second user via the network. One or more control tools is provided to the second user. The one or more control tools is configured to adjust one or more parameters of the virtual environment. A selection of at least one of the control tools is received from the second user. The virtual environment is adjusted based on the user selection.

In various embodiments, a computer program product for providing a virtual or augmented reality platform. The computer program product includes a computer readable storage medium having program instructions embodied therewith to perform a method where a virtual environment is provided to a first user via a virtual or augmented reality system at a first location. A first set of data is collected based on interaction of the first user with the virtual environment. The first set of data includes biometric data of the first user as the user engages in a training protocol received from a database. A real-time mirrored view of the virtual environment provided to the first user is provided to a second user via a network. The first set of data is provided to the second user via the network. One or more control tools is provided to the second user. The one or more control tools is configured to adjust one or more parameters of the virtual environment. A selection of at least one of the control tools is received from the second user. The virtual environment is adjusted based on the user selection.

### BRIEF DESCRIPTION OF THE SEVERAL VIEWS OF THE DRAWINGS

FIG. 1 illustrates an exemplary real-time Virtual Reality telecommunications systems (VRTS), in accordance with an embodiment of the present disclosure.

FIG. 2 illustrates an exemplary real-time VRTS, in accordance with an embodiment of the present disclosure.

FIG. 3A illustrates an exemplary real-time VRTS, in accordance with an embodiment of the present disclosure.

FIG. 3B illustrates an exemplary real-time VRTS, in accordance with an embodiment of the present disclosure.

FIG. 4 illustrates an exemplary system diagram of a real-time VRTS, in accordance with an embodiment of the present disclosure.

FIGS. 5A-5E illustrate exemplary interfaces for controlling and/or monitoring a user of a real-time VRTS, in accordance with an embodiment of the present disclosure.

FIG. 6 shows a flowchart of a method of treating a user using a real-time VRTS, in accordance with an embodiment of the present disclosure.

FIG. 7 illustrates an exemplary virtual reality headset according to embodiments of the present disclosure.

FIG. 8 illustrates an exemplary dialog system architecture using asynchronous messaging according to embodiments of the present disclosure.

FIG. 9 depicts a computing node according to an embodiment of the present invention.

### DETAILED DESCRIPTION

Currently, real-time virtual reality telecommunication software allows a user (e.g., a patient) to perform activities (e.g., training protocol, rehabilitation exercises, etc.) in a virtual environment, but does not allow any manual real-time manipulation of the environment by an outside user (e.g., a trainer or health care professional). Existing solutions do not accommodate the need to customize the environment to the specific needs of a user. Thus, there remains a need in the art for real-time telecommunication software that can be manipulated manually or automatically, so as to customize the environment to the specific needs of one or more users.

A “training protocol” may include, but is not limited to, a rehabilitation protocol, anesthesia replacement, cognitive training, neurological intervention, etc.

In various embodiments, the current disclosure provides a real-time Virtual Reality telecommunication system (VRTS). In various embodiments, the VRTS provides an immersive virtual environment to one or more users. In some embodiments, the VRTS enables one or more user to treat one or more other user using the VRTS. In some embodiments, the VRTS enables real-time data collection and/or treatment of one or more users interacting with a virtual environment provided by the VRTS.

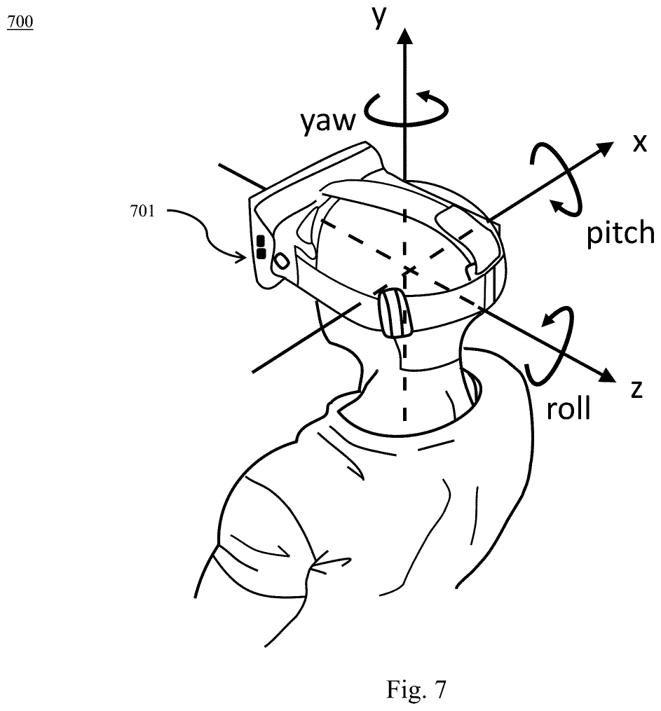

In various embodiments, the VRTS includes a data storage system, one or more cameras, and a communication system. The user(s) of the VRTS may use one or more sensors that can provide the VRTS information about the interaction of each user with the virtual environment provided by the VRTS. In some embodiments, the user(s) of the VRTS use a headset (for example, the headset described below with reference to FIG. 7) in communication with the VRTS to provide an immersive virtual environment to each user. In further embodiments, the user(s) can use interactive gloves, tools, and/or other controls with that allow for interaction with the virtual environment. In various embodiments, the user(s) may attach a mobile device to their body to collect data via, e.g., an internal gyroscopes and/or accelerometer.

In various embodiments, the VRTS may include a biofeedback process in which a user is provided information about a biometric measurement (e.g., a heart rate, breathing rate, brain electrical activity, etc.) as described in more detail below. In various embodiments, the user may be provided an instruction by the VRTS based on the biometric measurement. For example, the user may be instructed to maintain a predetermined heart rate (e.g., 70 bpm) for a predetermined amount of time. In another example the user may be instructed to raise or lower their heart rate to a target heart rate.

In various embodiments, additional sensors are included to measure characteristics of a subject in addition to motion. For example, cameras and microphones may be included to track speech, eye movement, blinking rate, breathing rate, and facial features. Biometric sensors may be included to measure features such as heart rate (pulse), inhalation and/or exhalation volume, perspiration, eye blinking rate, electrical activity of muscles, electrical activity of the brain or other parts of the central and/or peripheral nervous system, blood pressure, glucose, temperature, galvanic skin response, or any other suitable biometric measurement as is known in the art.

In various embodiments, an electrocardiogram (EKG) may be used to measure heart rate. In various embodiments, an optical sensor may be used to measure heart rate, for example, in a commercially-available wearable heart rate monitor device. In various embodiments, a wearable device may be used to measure blood pressure separately from or in addition to heart rate. In various embodiments, a spirometer may be used to measure inhalation and/or exhalation volume. In various embodiments, a humidity sensor may be used to measure perspiration. In various embodiments, a camera system may be used to measure the blinking rate of one or both eyes. In various embodiments, a camera system may be used to measure pupil dilation. In various embodiments, an electromyogram (EMG) may be used to measure electrical activity of one or more muscles. The EMG may use one or more electrodes to measure electrical signals of the one or more muscles. In various embodiments, an electroencephalogram (EEG) may be used to measure electrical activity of the brain. The EEG may use one or more electrodes to measure electrical signals of the brain. Any of the exemplary devices listed above may be connected (via wired or wireless connection) to the VR/AR systems described herein to thereby provide biometric data/measurements for analysis. In various embodiments, breathing rate may be measured using a microphone.

In various embodiments, a first user can interact, in real-time, with one or more additional users (e.g., a second user, a third user, a fourth user, etc.) connected to the VRTS. In some embodiments, one or more users can access collected data related to the first user. In some embodiments, one or more users can use collected data to provide one or more treatments, tasks, and/or information to the first user utilizing the VRTS. In some embodiments, a master user (e.g., a healthcare provider) may be able to monitor the virtual environment(s) of other users. In some embodiments, the VRTS may provide the master user a mirrored view of the other users. In some embodiments, the mirrored view may be identical to the view that another user is experiencing in the VRTS. In some embodiments, the mirrored view may be provided via a display on a tablet computer. In various embodiments, the mirrored view may be provided via a headset, such as the headset shown in FIG. 7.

In various embodiments, the master user (e.g., healthcare provider) may be located in close proximity to the one or more users (e.g., in the same room). In various embodiments, the master user may be wearing a headset (as described with respect to FIG. 7) with the one or more users. In various embodiments, the headset may provide the master user with the mirrored view of any one of the users. In various embodiments, the master user may switch between mirrored views of each user. In various embodiments, the master user may provide verbal commands to one or more of the users based on biometric data and/or the mirrored view.

In various embodiments, the master user may control one or more of the other user's virtual environments. In various embodiments, the collected data from other users may be displayed to the master user. In various embodiments, the collected data may be displayed to the master user in the VR interface (e.g., tablet display, headset display). In various embodiments, the master user may adjust parameters of the other users' virtual environments based on the collected data.

In various embodiments, the VRTS may provide treatments, tasks, or information, on a per user basis. For example, in one embodiment, a specific exercise may be shown and/or provided to one user while a different exercise may be shown to a second user. The VRTS may adjust each exercise as needed for each user to tailor the exercises to the specific user by collecting positional data over time for each user as they perform the exercises. In various embodiments, the adjustments may be manually implemented by the master user, e.g., an instructor or therapist.

It will be appreciated that a variety of virtual and augmented reality devices are known in the art. For example, various head-mounted displays providing either immersive video or video overlays are provided by various vendors. Some such devices integrate a smart phone within a headset, the smart phone providing computing and wireless communication resources for each virtual or augmented reality application. Some such devices connect via wired or wireless connection to an external computing node such as a personal computer. Yet other devices may include an integrated computing node, providing some or all of the computing and connectivity required for a given application.

Virtual or augmented reality displays may be coupled with a variety of motion sensors in order to track a user's motion within a virtual environment. Such motion tracking may be used to navigate within a virtual environment, to manipulate a user's avatar in the virtual environment, or to interact with other objects in the virtual environment. In some devices that integrate a smartphone, head tracking may be provided by sensors integrated in the smartphone, such as an orientation sensor, gyroscope, accelerometer, or geomagnetic field sensor. Sensors may be integrated in a headset, or may be held by a user, or attached to various body parts to provide detailed information on user positioning.

In various embodiments, additional sensors are included to measure characteristics of a subject in addition to motion. For example, cameras and microphones may be included to track speech and facial features. Biometric sensors may be included to measure features such as heart rate, blood pressure, glucose, temperature, or galvanic skin response.

In various embodiments, a user is furnished with a VR or AR system. As noted above, a VR or AR system will generally have integrated motion sensors. In addition, additional motions sensors may be provided, for example to be handheld. This allows tracking of multiple patient attributes while they interact with a scene. In this way, systematic and reproducible scenarios may be used to assess the subject's function.

In various embodiments, patient motion may be tracked. For example, Gait, Stability, Tremor, Amplitude of Motion, Speed of Motion, and Range of Motion may be measured. Movement may be analyzed to determine additional second order attributes such as smoothness or rigidity.

The tracking of these metrics allows the generation of quantified, detailed reports that are aligned with common practice evaluation procedures. It will be appreciated that a variety of evaluation protocol are known in the art.

In various embodiments, a master user may pair an external control device (e.g., a tablet computer) with one or more other user's VR/AR headset. In various embodiments, pairing includes an authentication protocol where the headset holds credentials that the external control device needs in order to validate the pairing process. For example, a headset may have an immutable identifier that must be provided by the external control device in order to pair. In another example, the headset may generate one-time keys that are used to authenticate a given external control device. In another example, a double opt-in process in used, in which both the headset user and the control device user concurrently opt-in to control. In various embodiments, only paired headsets will be able to connect to a specific external control interface.

In various embodiments, a master user may monitor one or more other users in their respective virtual environments. In various embodiments, the master user may be able to monitor the user experience in VR/AR using an external device (e.g., a tablet computer). In various embodiments, monitoring will enable the external observer to get status messages from the VR/AR headset such as: Experience status (Score/quality of experience/Time), Headset status (in terms of battery/connectivity/errors/current running application), and/or Patient status. In various embodiments, the clinician will be able to get patient status according to bio-feedback data in order to supply better care for the clinician. In various embodiments, the clinician may be able to stop the VR/AR session in extreme cases where bio-feedback data shows problematic results.

In various embodiments, the master user (e.g., health care provider) may communicate with the patient via a communications platform. In various embodiments, the communications platform may allow the master user to send messages to the patient (the master user may remotely communicate with the patient inside the VR environment). In various embodiments, the communications platform may allow the patient to send messages to the master user. In various embodiments, the messages may be text messages. In various embodiments, the messages may include a visual scale (e.g., a 1 to 10 scale, happy to sad scale, etc.). In various embodiments, the patient may mark their current state from different options on the visual scale. In various embodiments, the communications platform includes Voice Over IP and/or Video connection using video streaming features to enable better communication through the external control. In various embodiments, users in monitor state can control their interface independently and communicate with the master user, whether the master user is near or located on a different location, thus enabling tele-rehabilitation.

In various embodiments, the master user may be provided with a mirror view of one or more of the other users. In various embodiments, the master user may be able to mirror the user experience in VR/AR using an external device. In various embodiments, the mirrored view may cast the VR/AR experience in 2D video onto the external control device (e.g., tablet computer). In various embodiments, the mirror view (combined with the control features described in more detail below) creates value for the master user by providing access to the full capacity of user experience in VR/AR. In various embodiments, the mirrored view enables the master user (e.g., a therapist) to see the patient movements and results in real time and take an active role in the training process. In various embodiments, the master user may receive patient results, change session parameters, and/or help the patient adjust his training as if the clinician was in the same room.

In various embodiments, the master user may be provided with control tools to modify parameters associated with each user's virtual environment. In various embodiments, the master user may control the user experience in VR/AR using an external device (e.g., a tablet computer). In various embodiments, the master user may send control messages to one or more user's headset(s) and/or receive status updates from the one or more user's headset(s). In various embodiments, control messages may include: initiating experience in VR/AR, stopping an experience in VR/AR, setting experience parameters in VR/AR, and/or changing experience parameters in real-time according to user performance and results sent back to the external control interface. In various embodiments, the master user will be able to use bio-feedback data to control patient experience accordingly inside VR/AR. In various embodiments, control tools may be enabled manually according to bio-feedback data presented to the clinician or by an algorithm changing user experience automatically according to bio-feedback data.

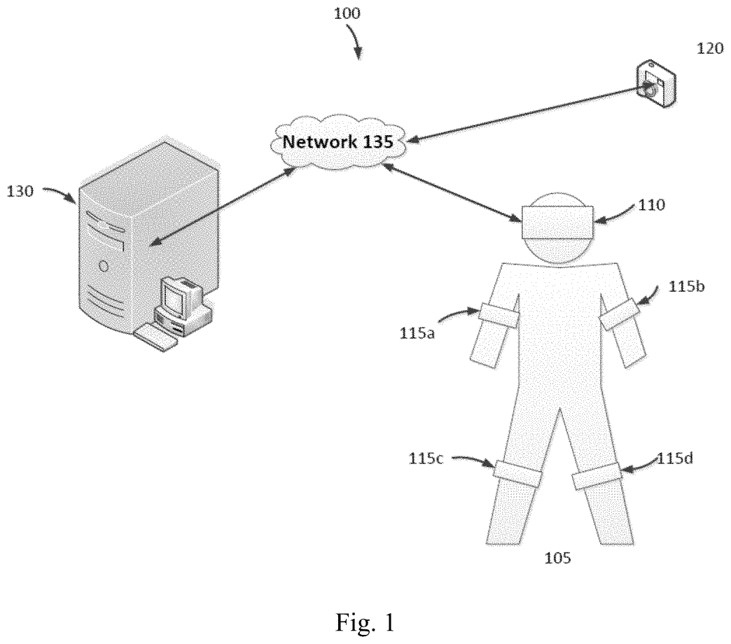

FIG. 1 illustrates an exemplary real-time Virtual Reality telecommunications systems (VRTS) 100, in accordance with embodiments of the present disclosure. As shown in FIG. 1, the VRTS 100 includes a data storage system 130, a network 135, a camera 120, sensors (115a-115d, 115, generally), and a Virtual Reality (VR) headset 110. The user 105 is wearing sensors 115 can provide information about the user 105 to the data storage system 130. In addition, camera 120 can provide motion data of the user. In many embodiments, the VRTS may use one or more data storage systems, one or more sensors, and one or more cameras to monitor, record, and/or track data related to the user. In this embodiment, each portion of the VRTS 100 is in communication with other portions of the VRTS 100 through a wireless connection. In some embodiments, one or more portions may be connected using wired connections.

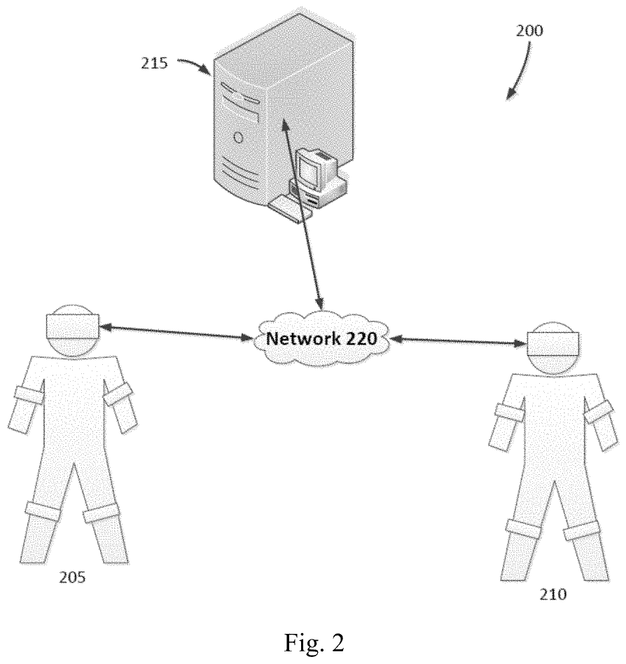

FIG. 2 illustrates an exemplary real-time VRTS 200, in accordance with embodiments of the present disclosure. In some such embodiments, the VRTS 200 includes data storage system 215, network 220, user 205 and user 210. User 205 and user 210 are interacting with each other within a virtual environment provided by data storage system 215. Each portion of the VRTS 200 are in communication with each other portion of the VRTS 200 using network 220. In this embodiment, network 220 represents a wireless network through which data from user 205 and user 210 is collected and/or stored by data storage system 215. In various embodiments, user 210 is enabled to access data stored on the data storage system 215, and can affect the virtual environment by modifying one or more parameters of the virtual environment.

In various embodiments, the VRTS 200 provides a virtual environment to users 205, 210 using the data storage system 215. Data Storage System 215 collects data from both user 205 and user 210 using sensors attached to users 205 and user 210. Data Storage system 215 is enabled to analyze the collected data to determine a first treatment. In some embodiments, the user(s) may be enabled to analyze data and prescribe one or more treatments to another user based on the data. Data storage system 215 implements the first treatment by layering the first treatment over the virtual environment provided by the data storage system 215. The data storage system 215 collects data related to user 205 and the first treatment. In various embodiments, the data storage system 215 analyzes the collected data. In various embodiments, the data storage system 215 may applies one or more adjustments to the virtual environment and/or treatment based on input from a master user.

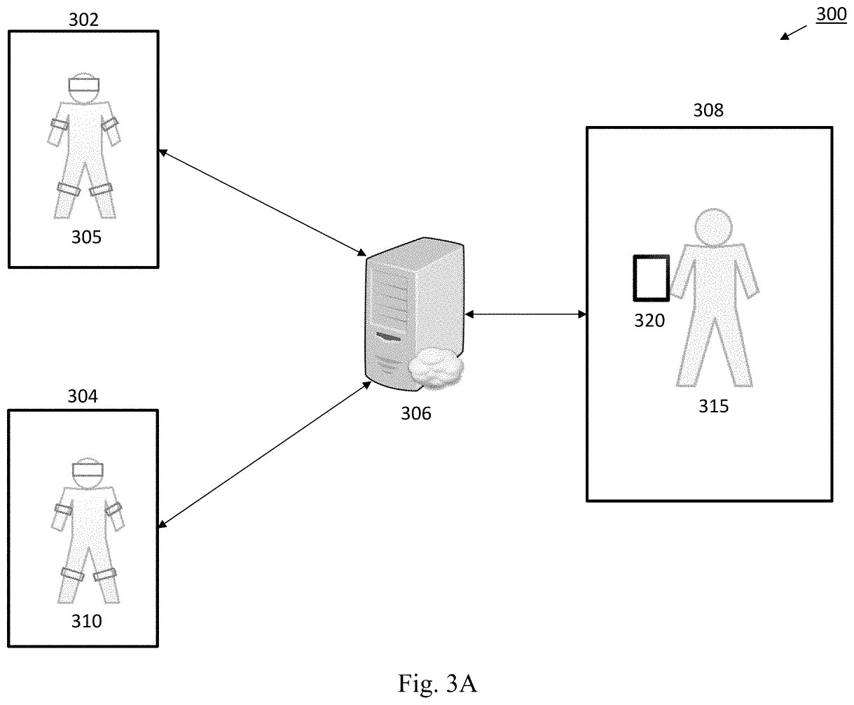

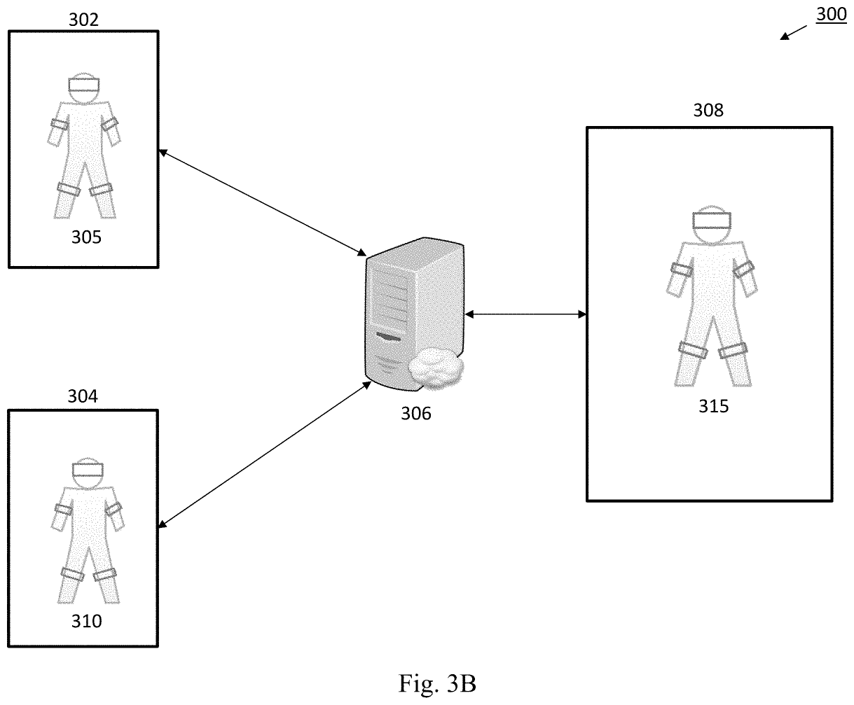

FIG. 3A illustrates an exemplary real-time VRTS 300, in accordance with an embodiment of the present disclosure. The VRTS 300 includes a first user 305 at a first location 302 and a second user 310 at a second location 304. In various embodiments, the first location 302 and the second location 304 may be different locations (e.g., each user's residence). In various embodiments, the first location 302 and the second location 304 may be the same locations (e.g., a common space accessible to multiple users). The first user 305 and the second user 310 may be wearing the VR/AR headset and/or motion tracking sensors as described in more detail with respect to FIG. 7.

In various embodiments, the VRTS 300 includes a third user 315 (e.g., a master user) at a third location 308. In various embodiments, the third user 315 may use a tablet computer to monitor and/or control the virtual environments of the first user 305 and/or second user 310. In various embodiments, the third user 315 may be an instructor, trainer, physical therapist, or other healthcare provider.

In various embodiments, computer nodes implementing the VRTS 300 for the first user 305, the second user 310, and the third user 315 are connected via a network to one or more remote servers 306. In various embodiments, the remote server 306 may be a cloud server. In various embodiments, the remote server 306 may be located at the location of, e.g., the instructor or company providing the treatment or assessment protocols. In various embodiments, the one or more servers 306 may include a database, such as, for example, an EHR database.

In various embodiments, the third user 315 may receive data (e.g., biometric data, positional data, video data, and/or audio data) from the first user 305 and/or the second user 310. In various embodiments, positional data of the first user 305 and/or the second user 310 may be recorded by sensors attached to the body and sent via the network to the third user 315. In various embodiments, one or more users may be shown the same protocol. In various embodiments, one or more users may be shown a different protocol.

In various embodiments, the protocol is received from a healthcare record server. In various embodiments, the healthcare record server has a database for storing electronic health records. In various embodiments, an electronic health record of the user may be accessed to retrieve one or more parameters related to the protocol.

In various embodiments, the VRTS may measure and/or record one or more biometric measurement. In various embodiments, the biometric measurement is selected from: heart rate, blood pressure, breathing rate, electrical activity of the muscles, electrical activity of the brain, pupil dilation, and perspiration.

In various embodiments, the biometric measurement may be transmitted to the master user. In various embodiments, the biometric measurement may be presented to the master user (e.g., on a display or in a specific virtual environment for the master user).

In various embodiments, when the biometric measurement is outside of a predetermined range or above/below a predetermined threshold, the VRTS 300 may provide an indication to the third user 315 that the biometric measurement is out of the range. In various embodiments, the out-of-range biometric measurement and/or the user may be highlighted to notify the third user 315 of the out-of-range measurement. In this example, the third user may instantiate a mirroring/control session (if one hasn't already been started) with the particular user having an out-of-range measurement. In various embodiments, the third user 315 may use the control tools provided to them by the VRTS 306 to adjust one or both of the virtual environment and the training protocol. In various embodiments, the third user 315 may adjust a difficulty of the training protocol. For example, the third user 315 may increase the number of reps in a training protocol if a biometric measurement (e.g., heart rate) is not above a predetermined threshold.

In various embodiments, the virtual environments provided to the first user 305 and the second user 310 do not have any control interfaces and the third user 315 has sole authority to observe each user via the mirrored view and/or adjust the virtual environment via the control tools.

FIG. 3B illustrates an exemplary real-time VRTS, in accordance with an embodiment of the present disclosure. Similar to FIG. 3A, the VRTS 300 includes a first user 305 at a first location 302 and a second user 310 at a second location 304, and a third user 315 (e.g., a master user) at a third location 308. In various embodiments, the third user may be wearing the VR/AR headset and/or motion tracking sensors as described in more detail with respect to FIG. 7.

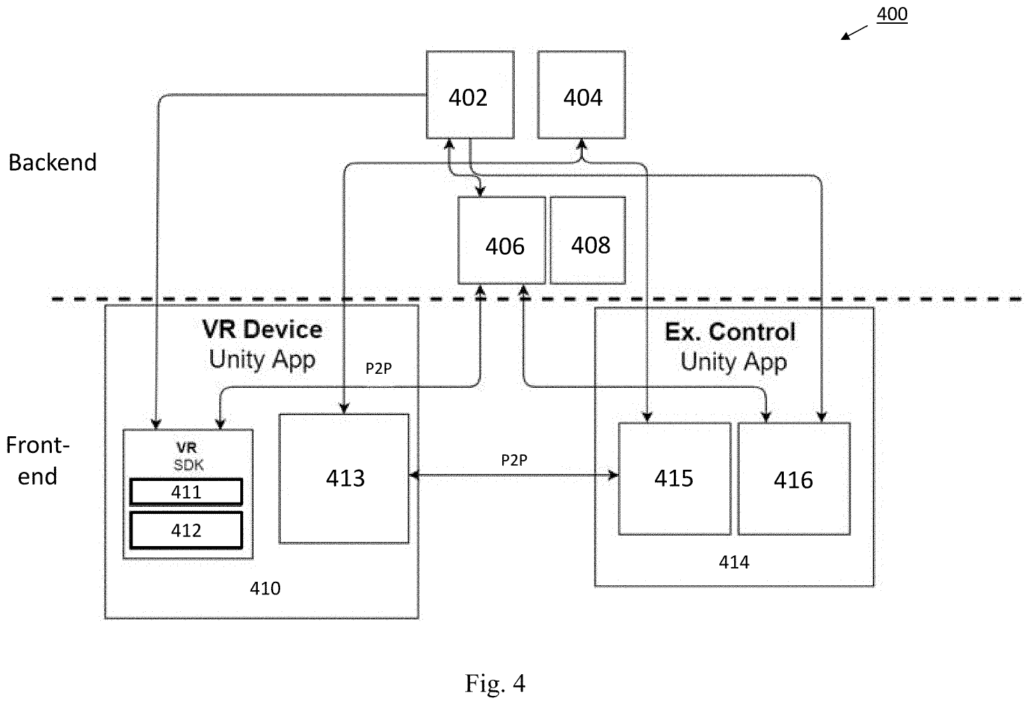

FIG. 4 illustrates an exemplary system diagram 400 of a real-time VRTS, in accordance with an embodiment of the present disclosure. In various embodiments, the system 400 includes a backend having various modules, such as, for example, asynchronous messaging architectures 402, 404, functions 406, and/or tables 408. In various embodiments, the backend may include a serverless interface to move messages from the external control to the VR/AR device and back. In various embodiments, the backend may include one or more database to store pairing data and authenticate between the external control and the VR/AR device. In various embodiments, the backend may include a service that allows server code to send asynchronous notifications to client-side web applications (e.g., push messages) to enable the headset get messages initiated by the external control interface. In various embodiments, the backend may include a video recorder to enable mirroring and/or send-by-streaming. In various embodiments, the backend may include an audio recorder and/or send-over-IP in order to enable vocal communication between the master user and the patient.

In various embodiments, asynchronous messaging is a communication method where a message is placed in a message queue and does not require immediate processing to continue operating a program. In various embodiments, examples include a request for information, explanation, or data needed (but not needed immediately). In various embodiments, an asynchronous messaging system may be used to transmit data to a system for processing where the data may be placed in a queue at a server for processing. In various embodiments, asynchronous messaging may be called fire-and-forget information exchange or message-oriented middleware (MOM).

In various embodiments, the system 400 further includes a front end that includes a VR device unity app 410 and an External Control unity app 414. The VR device unity app 410 includes a VR SDK module that may include one or more VR modules 411 and/or game engines 412. The VR device unity app 410 may also include a video recorder/streaming module 413. The External Control unity app 414 includes player module 415 and an External Control SDK 416. As shown in FIG. 4, the modules may communicate with one another via a communications protocol as is known in the art (e.g., P2P). In various embodiments, the front-end may be generated via any suitable front-end technology, such as, for example, Web/Android/IOS. In various embodiments, the front end may be implemented using a Unity Game engine.

In some embodiments, separate communication channels are provided for control signals and for video. For example, in some embodiments, a video recorder is collocated with the VR player on the VR device. The recorder records the VR experience as it is being displayed to a user. In some embodiments, the recorded video is streamed to a player, provided as part of the controller via a peer-to-peer connection. In some embodiments a signaling server, such as a n asynchronous messaging signaling server, acts to coordinate the peer-to-peer video connection between the recorder and the player. For example, in some embodiments, the signaling server handles messages including session control messages used to open or close communication between recorders and players, error messages, media metadata such as codecs and codec settings, data used to establish secure connections, and network data.

In some embodiments, a separate messaging server is provided for handling control messages. For example, in some embodiments, an asynchronous messaging architecture is used. The messaging server can send and receive control messages to and from the controller and the VR device, including the control messages set out herein.

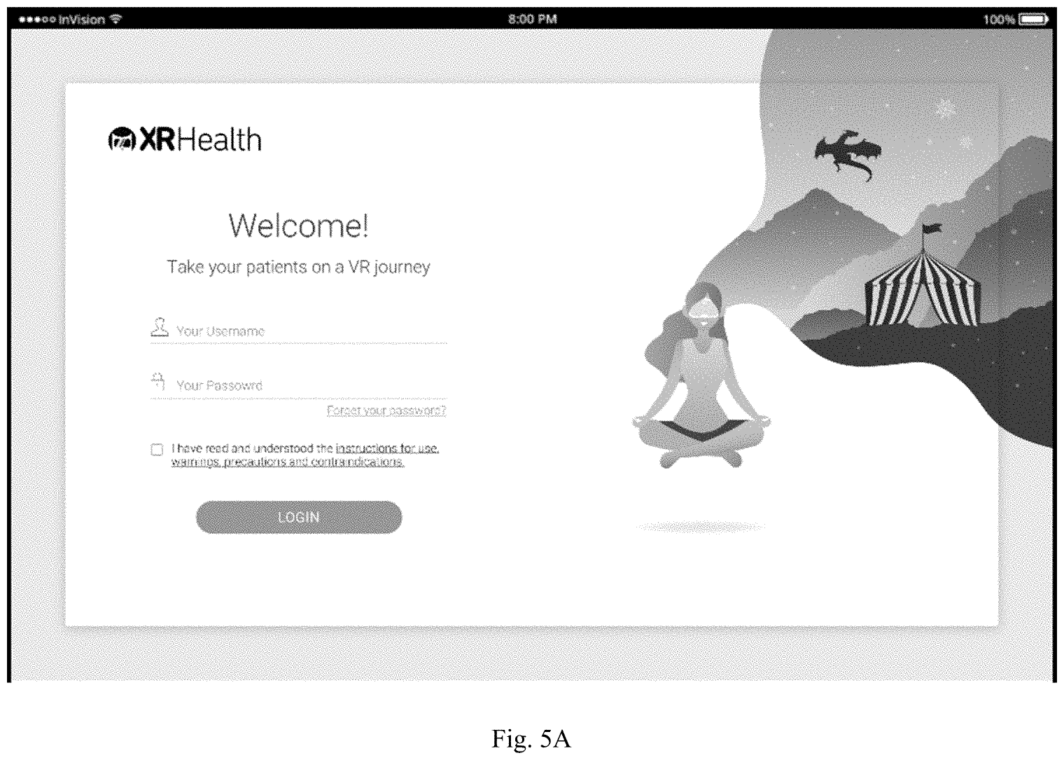

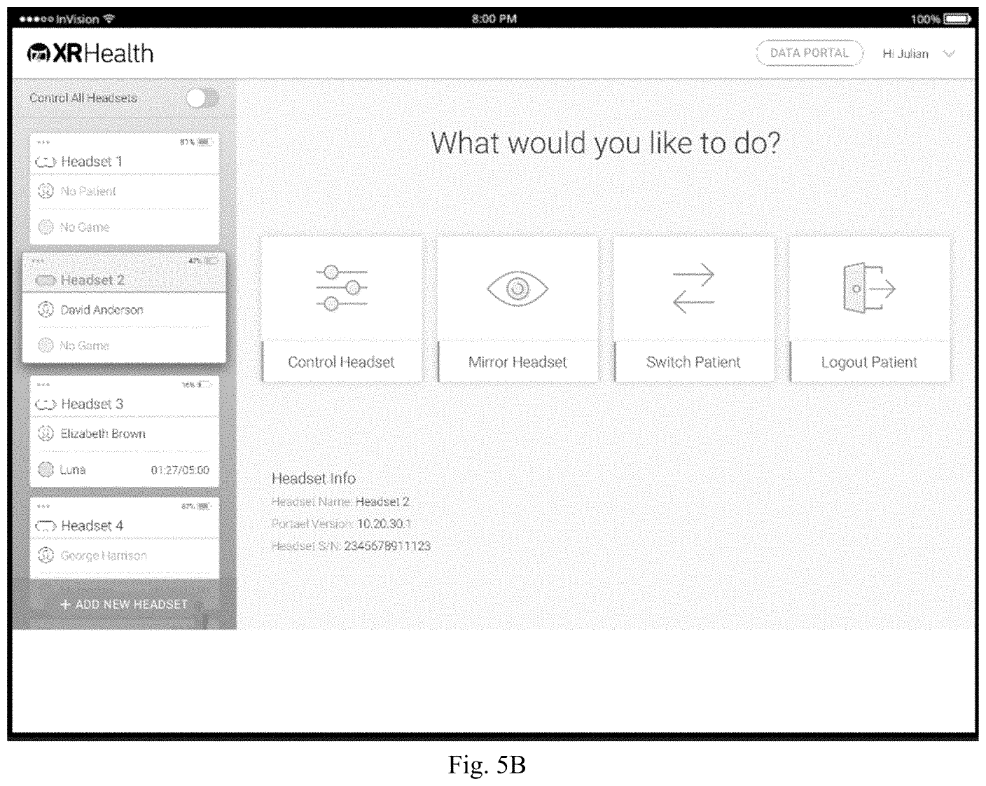

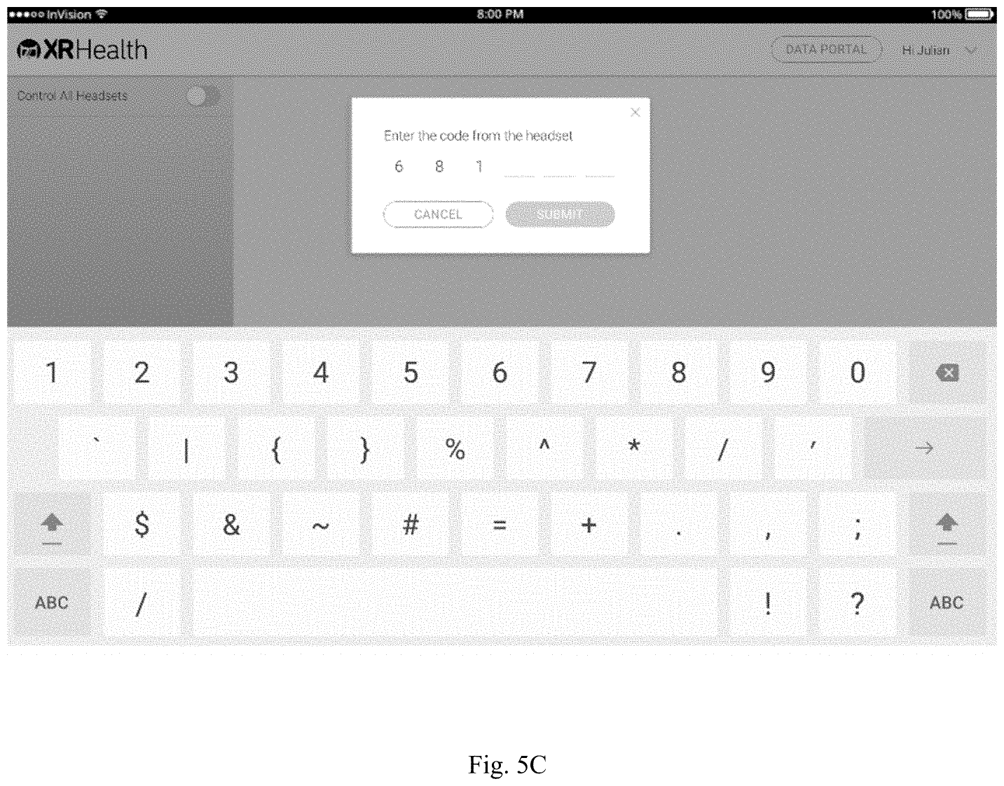

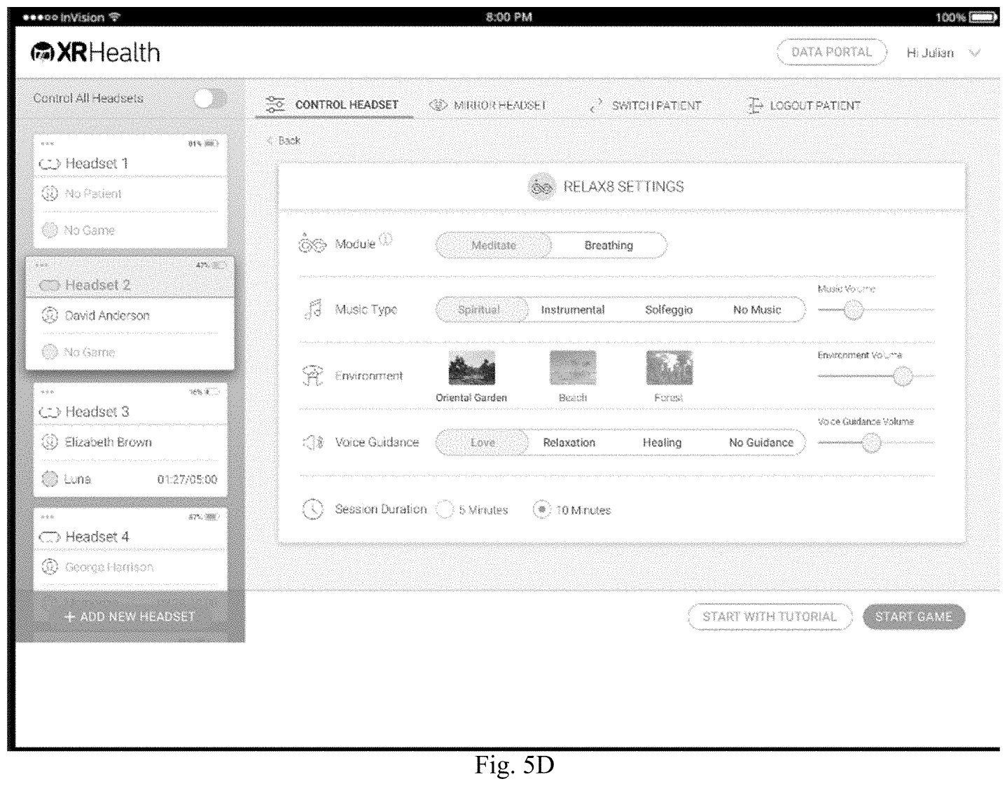

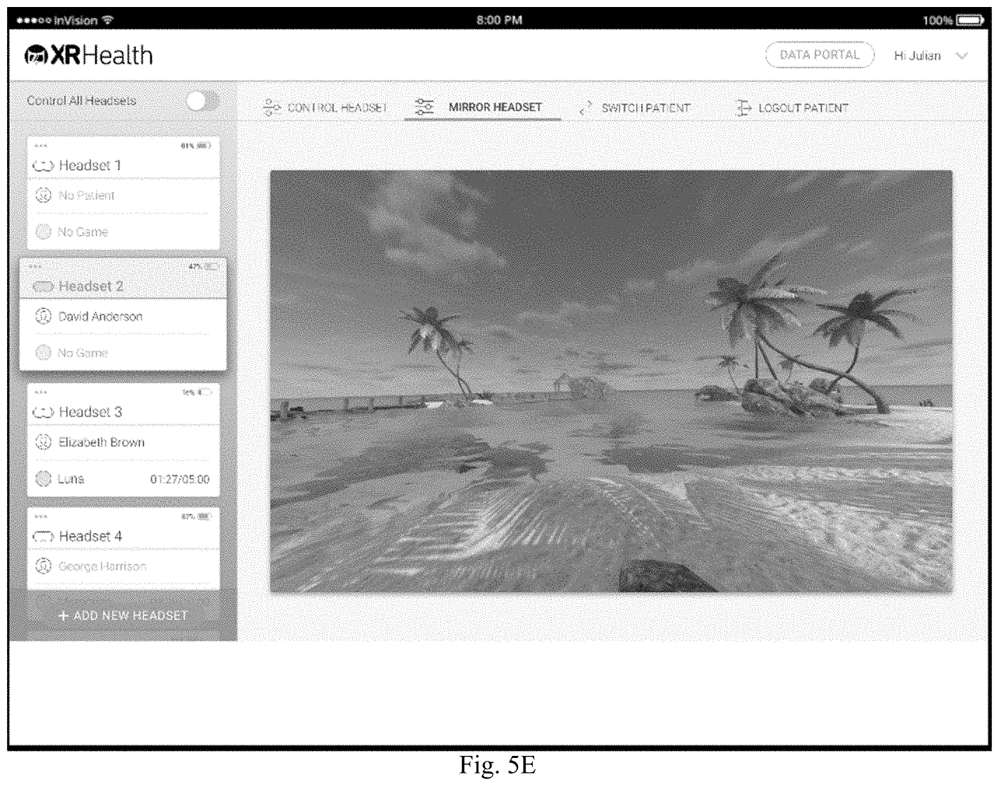

FIGS. 5A-5E illustrate exemplary interfaces for controlling and/or monitoring a user of a real-time VRTS, in accordance with an embodiment of the present disclosure. FIG. 5A illustrates an exemplary log-in screen for a master user to access control tools. FIG. 5B illustrates an exemplary landing page for the control interface for a master user. In particular, the interface includes four users (headsets 1-4) who are paired with the control interface. In various embodiments, the master user may be presented with various data about each user and/or the equipment they are using (e.g., remaining battery, name, sensors, biometric data, training protocol, remaining time, etc.). In various embodiments, the master user may control any individual headset using the “Control Headset” option. The master user may opt to control all headsets via a toggle button. In various embodiments, the master user may mirror the view of any individual headset using the “Mirror Headset” option. In various embodiments, the master user may switch to controlling another patient using the “Switch Patient” option. In various embodiments, the master user may log a patient out of the virtual environment using the “Logout Patient” option. In various embodiments, the control interface may indicate to the master user what particular training protocol each user is being presented (if any). In various embodiments, the control interface may indicate a remaining time that a particular user has to finish the training protocol. FIG. 5C illustrates pairing a headset with the control interface using a six-digit code.

FIG. 5D illustrates various control tools included in the control interface. In various embodiments, the master user may change a training module or protocol that a particular user is following. For example, the master user may switch another user from a meditation module to a breathing module. In various embodiments, the master user may adjust a particular user's module based on biometric data provided to the control interface from each user. In various embodiments, the master user may change sounds that a particular user is hearing. For example, the master user may select a music type such as spiritual, instrumental, solfeggio, or no music. In various embodiments, the master user may also control the volume of the sounds for each user. In various embodiments, the master user may control the virtual environment that each user experiences. For example, the master user may select a virtual environment for each user from the options of: an oriental garden, a beach, and a forest. In various embodiments, the master user may control the environment volume. In various embodiments, the master user may adjust the voice guidance. For example, the master user may select from love, relaxation, healing, or no guidance. In various embodiments, the master user may adjust the volume of the voice guidance. In various embodiments, the master user may control a session duration. For example, the master user may select a session duration of 5 minute or 10 minutes for each user. In this example, each user may have the same session duration, or may have different session durations. In various embodiments, a custom session duration may be entered. Any of the features described above may be adjusted in response to biometric data that is received at the control interface.

In various embodiments, the users are not provided with the capability to change these features of the virtual environment—only the master user may be provided access to the control interface.

FIG. 5E illustrates a mirrored view of the view from one of the users (e.g., headset 2) at the control interface. The mirrored view shows exactly what the particular user sees in their headset. In this example, the person wearing headset 2 is viewing a beach.

It will be appreciated that the present disclosure is useful to a variety of users in a variety of contexts. For example, a first user may be a healthcare provider, coach, or trainer, while a second user may be a patient, athlete, or trainee. Based on real-time analysis of user performance, quantified reports may be provided according to common practice evaluation procedures. The first user may then customize the training or treatment regimen and provide one or more additional layers of output in the virtual environment for the second user. In this way, a customized virtual training or treatment session is provided. Ongoing monitoring and analysis may be provided, and the data may be provided to both users. Continuous, real-time communication is provided among the users.

In various embodiments, the VRTS may provide the data of the first user to a first learning system. In various embodiments, the first learning system may be trained based on, for example, a data set of user data during rehabilitation exercises. In various embodiments, the first trained learning system may receive the user biometric data and/or the training protocol as input. In various embodiments, the first trained learning system may output an adjustment of one or more parameters of the virtual environment. For example, the adjustment may increase the speed of visual cues for a user to perform an action (e.g., a punching motion). In another example, the adjustment may decrease the amount of time a visual cue is present for the user to hold a particular position (e.g., for a stretching exercise).

In various embodiments, the adjustment may be provided to a second trained learning system. In various embodiments, the second trained learning system may receive the adjustment, user biometric data, and/or training protocol. In various embodiments, the second learning system may output a predicted response of the user based on the adjustment, user biometric data, and/or training protocol. In various embodiments, the predicted response may be a prediction of how one or more biometric data will change given the adjustment to the virtual environment of the user. In various embodiments, the predicted response may be an increase in the one or more biometric data (e.g., heart rate) when the adjustment is applied to the virtual environment of the user. In various embodiments, the predicted response may be a decrease in the one or more biometric data (e.g., heart rate) when the adjustment is applied to the virtual environment of the user. In various embodiments, the predicted response may be minimal change (e.g., no change) in the one or more biometric data (e.g., heart rate) when the adjustment is applied to the virtual environment of the user.

In various embodiments, the VRTS may determine whether the predicted response passes a predetermined threshold. In various embodiments, the predetermined threshold may be received from an electronic health record (EHR) database. In various embodiments, the EHR database may be the same database in which the training protocol is stored. In various embodiments, the predetermined threshold may be contained within the training protocol. In various embodiments, the predetermined threshold may be a target value for one or more biometric data (e.g., heart rate). For example, the VRTS system may determine an adjustment from the first learning system that speeds up a visual cue that instructs the user to perform an exercise (e.g., a jumping jack). In this example, VRTS system then determines a predicted response of the user at the second learning system, which may be that the user heart rate increase by 15 beats per minute (bpm) from 90 bpm to 105 bpm. If the target heart rate is 100 bpm, the system may provide an indication to an instructor that the particular adjustment to the virtual environment will likely result in the target heart rate being met or exceeded.

In various embodiments, when the biometric measurement is determined to meet or exceed the predetermined threshold in the predicted response of the user, the adjustment may be provided to an instructor who is overseeing a physical therapy and/or rehabilitation session. In various embodiments, the instructor may be provided with one or more proposed adjustments along with the predicted response for each scenario. In various embodiments, an indication may be provided to indicate to the instructor whether the predicted response will meet or pass the predetermined threshold.

In some embodiments, a feature vector is provided to a learning system. Based on the input features, the learning system generates one or more outputs. In some embodiments, the output of the learning system is a feature vector.

In some embodiments, the learning system comprises a SVM. In other embodiments, the learning system comprises an artificial neural network. In some embodiments, the learning system is pre-trained using training data. In some embodiments training data is retrospective data. In some embodiments, the retrospective data is stored in a data store. In some embodiments, the learning system may be additionally trained through manual curation of previously generated outputs.

In some embodiments, the learning system, is a trained classifier. In some embodiments, the trained classifier is a random decision forest. However, it will be appreciated that a variety of other classifiers are suitable for use according to the present disclosure, including linear classifiers, support vector machines (SVM), or neural networks such as recurrent neural networks (RNN).

Suitable artificial neural networks include but are not limited to a feedforward neural network, a radial basis function network, a self-organizing map, learning vector quantization, a recurrent neural network, a Hopfield network, a Boltzmann machine, an echo state network, long short term memory, a bi-directional recurrent neural network, a hierarchical recurrent neural network, a stochastic neural network, a modular neural network, an associative neural network, a deep neural network, a deep belief network, a convolutional neural networks, a convolutional deep belief network, a large memory storage and retrieval neural network, a deep Boltzmann machine, a deep stacking network, a tensor deep stacking network, a spike and slab restricted Boltzmann machine, a compound hierarchical-deep model, a deep coding network, a multilayer kernel machine, or a deep Q-network.

Artificial neural networks (ANNs) are distributed computing systems, which consist of a number of neurons interconnected through connection points called synapses. Each synapse encodes the strength of the connection between the output of one neuron and the input of another. The output of each neuron is determined by the aggregate input received from other neurons that are connected to it. Thus, the output of a given neuron is based on the outputs of connected neurons from preceding layers and the strength of the connections as determined by the synaptic weights. An ANN is trained to solve a specific problem (e.g., pattern recognition) by adjusting the weights of the synapses such that a particular class of inputs produce a desired output.

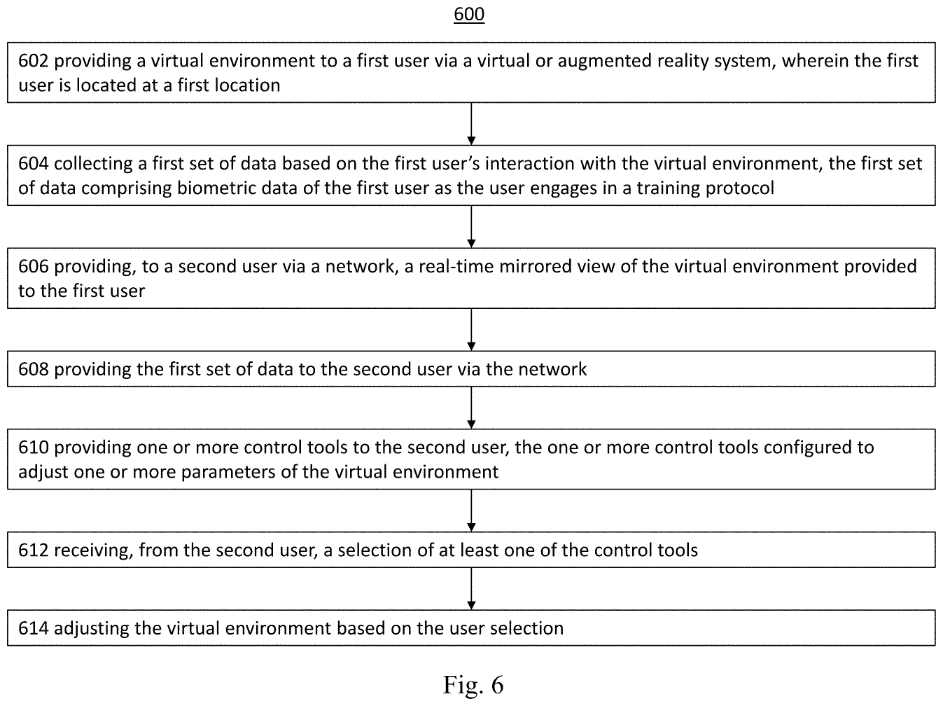

FIG. 6 shows a flowchart of a method 600 for control and monitoring of a virtual or augmented reality environment provided to patients. At 602, a virtual environment is provided to a first user via a virtual or augmented reality system at a first location. At 604, a first set of data is collected based on the first user's interaction with the virtual environment. The first set of data includes biometric data of the first user as the user engages in a rehabilitation protocol. At 606, a real-time mirrored view of the virtual environment provided to the first user is provided to a second user via a network. At 608, first set of data is provided to the second user via the network. At 610, one or more control tools is provided to the second user. The one or more control tools is configured to adjust one or more parameters of the virtual environment. At 612, a selection of at least one of the control tools is received from the second user. At 614, the virtual environment is adjusted based on the user selection

In various embodiments, the activity is a treatment protocol. In various embodiments, the activity is an assessment protocol. In various embodiments, the activity is a rehabilitation protocol. In various embodiments, a third set of data may be collected for a third user. The third set of data may include positional data. In various embodiments, the first activity may be displayed to the third user by layering the first activity over the virtual environment. In various embodiments, a fourth set of data is collected related to the third user and the first activity in the virtual environment. The fourth set of data may include positional data of the third user during the activity. In various embodiments, a second adjustment may be applied to the first activity for the third user. The second adjustment may be based on the fourth set of data. In various embodiments, the second adjustment may be the same or different than the first adjustment.

With reference now to FIG. 6, an exemplary virtual reality headset is illustrated according to embodiments of the present disclosure. In various embodiments, system 600 is used to collected data from motion sensors including hand sensors (not pictured), sensors included in headset 601, and additional sensors such as sensors placed on the body (e.g., torso, limbs, etc.) or a stereo camera. In some embodiments, data from these sensors is collected at a rate of up to about 150 Hz. As illustrated, data may be collected in six degrees of freedom: X—left/right; Y—up/down/height; Z—foreword/backward; P—pitch; R—roll; Y—yaw. As set out herein, this data may be used to track a user's overall motion to facilitate interaction with a virtual environment and to evaluate their performance. Pitch/Roll/Yaw may be calculated in Euler angles.

In various embodiments, off the shelf VR systems are optionally used with additional external compatible sensors to track various elements in multiple fields including, e.g., motion tracking, cognitive challenges, speech recognition, stability, facial expression recognition, and biofeedback.

Motion tracking can include, but is not limited to tracking of gait, stability, tremors, amplitude of motion, speed of motion, range of motion, and movement analysis (smoothness, rigidity, etc.).

Cognitive challenges can include, but is not limited to reaction time, success rate in cognitive challenges, task fulfillment according to different kind of guidance (verbal, written, illustrated, etc.), understanding instructions, memory challenges, social interaction, and problem solving.

Speech Recognition can include, but is not limited to fluent speech, ability to imitate, and pronunciation.

Stability can include, but is not limited to postural sway.

Bio-Feedback can include, but is not limited to, Heart rate variability (HRV),

Electrothermal activity (EDA), Galvanic skin response (GSR), Electroencephalography (EEG), Electromyography (EMG), Eye tracking, Electrooculography (EOG), Patient's range of motion (ROM), Patient's velocity performance, Patient's acceleration performance, and Patient's smoothness performance.

A Picture Archiving and Communication System (PACS) is a medical imaging system that provides storage and access to images from multiple modalities. In many healthcare environments, electronic images and reports are transmitted digitally via PACS, thus eliminating the need to manually file, retrieve, or transport film jackets. A standard format for PACS image storage and transfer is DICOM (Digital Imaging and Communications in Medicine). Non-image data, such as scanned documents, may be incorporated using various standard formats such as PDF (Portable Document Format) encapsulated in DICOM.

An electronic health record (EHR), or electronic medical record (EMR), may refer to the systematized collection of patient and population electronically-stored health information in a digital format. These records can be shared across different health care settings. Records may be shared through network-connected, enterprise-wide information systems or other information networks and exchanges. EHRs may include a range of data, including demographics, medical history, medication and allergies, immunization status, laboratory test results, radiology images, vital signs, personal statistics like age and weight, and billing information.

EHR systems may be designed to store data and capture the state of a patient across time. In this way, the need to track down a patient's previous paper medical records is eliminated. In addition, an EHR system may assist in ensuring that data is accurate and legible. It may reduce risk of data replication as the data is centralized. Due to the digital information being searchable, EMRs may be more effective when extracting medical data for the examination of possible trends and long term changes in a patient. Population-based studies of medical records may also be facilitated by the widespread adoption of EHRs and EMRs.

Health Level-7 or HL7 refers to a set of international standards for transfer of clinical and administrative data between software applications used by various healthcare providers. These standards focus on the application layer, which is layer 7 in the OSI model. Hospitals and other healthcare provider organizations may have many different computer systems used for everything from billing records to patient tracking. Ideally, all of these systems may communicate with each other when they receive new information or when they wish to retrieve information, but adoption of such approaches is not widespread. These data standards are meant to allow healthcare organizations to easily share clinical information. This ability to exchange information may help to minimize variability in medical care and the tendency for medical care to be geographically isolated.

In various systems, connections between a Picture Archiving and Communication System (PACS), Electronic Medical Record (EMR), Hospital Information System (HIS), Radiology Information System (RIS), or report repository are provided. In this way, records and reports form the EMR may be ingested for analysis. For example, in addition to ingesting and storing HL7 orders and results messages, ADT messages may be used, or an EMR, RIS, or report repository may be queried directly via product specific mechanisms. Such mechanisms include Fast Health Interoperability Resources (FHIR) for relevant clinical information. Clinical data may also be obtained via receipt of various HL7 CDA documents such as a Continuity of Care Document (CCD). Various additional proprietary or site-customized query methods may also be employed in addition to the standard methods.

In various embodiments, the systems described herein may be used to provide telehealth services. In various embodiments, an external controller (and the instructor) may be located at a remote location away from one or more (e.g., all) of the users. In various embodiments, the external controller may be implemented via a tablet, mobile device, personal computer, laptop, and/or a virtual or augmented reality system (such as a commercially available VR/AR system).

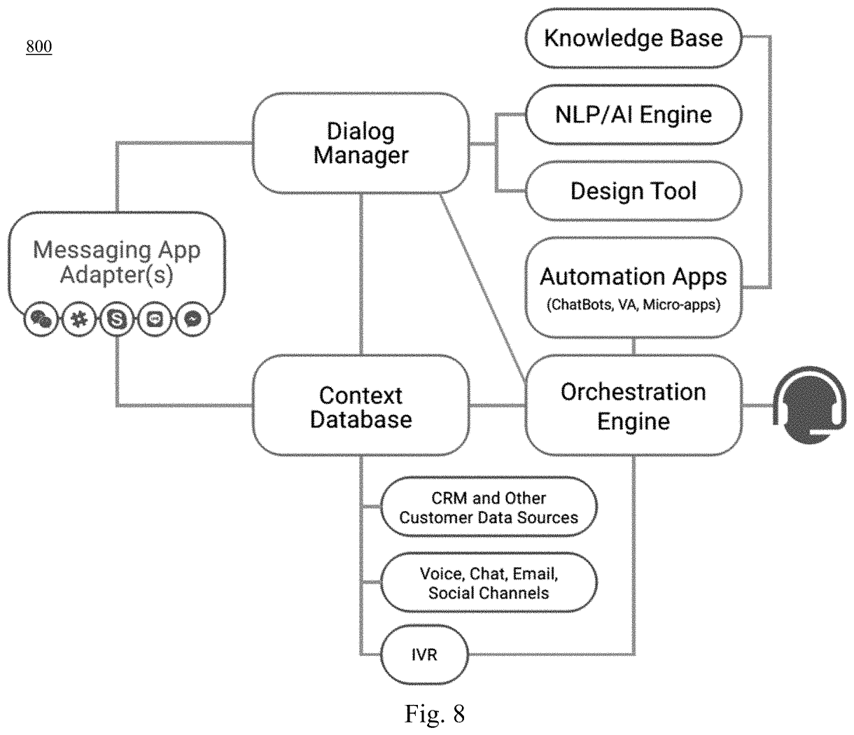

FIG. 8 illustrates an exemplary dialog system architecture 800 using asynchronous messaging. In various embodiments, the asynchronous messaging dialog system architecture 800 may include a blended AI (e.g., part AI responses, part human responses if AI does not complete the interaction with the user). In various embodiments, the architecture 800 includes one or more messaging adapter(s) to exchange text, media, and metadata with messaging apps/platforms. In various embodiments, the architecture 800 includes a dialog manager that controls the flow of the conversation between different actors, such as directed dialog, chatbots/AI systems, and/or connections to live representatives. In various embodiments, the architecture 800 includes one or more context database(s) for context on all interactions relevant to customer journeys. In various embodiments, the architecture 800 includes one or more design tools that function as the design environment used to build dialog flows, build chatbots, define conversation types, configure session length, and configure business rules to determine tasks/next steps in dialogs. In various embodiments, the architecture 800 includes one or more automation apps that include one or more chatbots built in environment or connected to the dialog system. In various embodiments, the architecture 800 includes an orchestration engine such as a stateful routing engine that conducts the flow of the conversation between different components, including, for example, web service and RESTful interface to engage other applications and data sources as needed. In various embodiments, the architecture 800 includes one or more natural language processing (NLP) and/or artificial intelligence engine(s) for intent analysis. In various embodiments, the architecture 800 includes one or more knowledge base(s) that is a central repository of customer facing information. In various embodiments, the architecture 800 includes one or more voice/chat/email/social engine(s) so that conventional channels may be incorporated for context and escalation. In various embodiments, the architecture 800 includes one or more Intelligent Voice Response System(s) (IVR) incorporated for consistent context and ability to use established application logic and backend integrations. In various embodiments, the architecture 800 includes one or more customer relationship management database and/or other enterprise data store(s) as necessary to ensure access to relevant customer data.

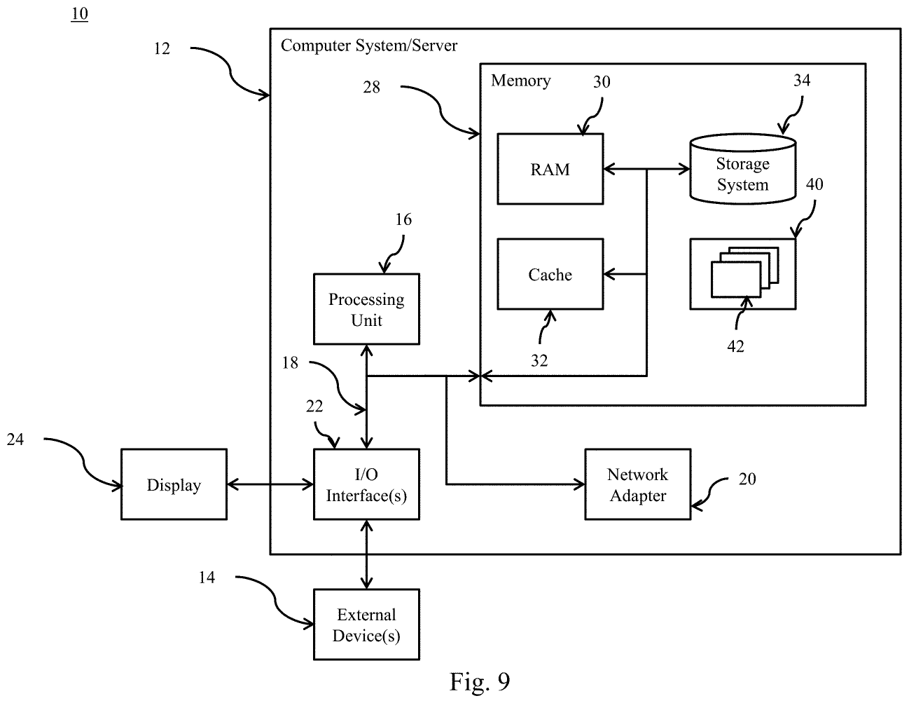

Referring now to FIG. 9, a schematic of an example of a computing node is shown. Computing node 10 is only one example of a suitable computing node and is not intended to suggest any limitation as to the scope of use or functionality of embodiments of the invention described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 9, computer system/server 12 in computing node 10 is shown in the form of a general-purpose computing device. The components of computer system/server 12 may include, but are not limited to, one or more processors or processing units 16, a system memory 28, and a bus 18 that couples various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the invention.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments of the invention as described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A method comprising:
providing a virtual environment to a first user via a virtual or augmented reality system, wherein the first user is located at a first location;
collecting a first set of data based on interaction of the first user with the virtual environment, the first set of data comprising biometric data of the first user as the user engages in a training protocol received from a database;
providing, to a second user via a network, a real-time mirrored view of the virtual environment provided to the first user;
providing the first set of data to the second user via the network;
providing one or more control tools to the second user, the one or more control tools configured to adjust one or more parameters of the virtual environment;
receiving, from the second user, a selection of at least one of the control tools; and
adjusting the virtual environment based on the user selection.

2. The method of claim 1, wherein the mirrored view is provided via a second virtual or augmented reality system.

3. The method of claim 1, wherein the mirrored view is provided via a display of a tablet computer.

4. The method of claim 1, wherein the first user does not have access to the one or more control tools for the virtual environment.

5. The method of claim 1, wherein the training protocol comprises a rehabilitation protocol, anesthesia replacement, cognitive training, and/or neurological intervention.

6. The method of claim 1, wherein the real-time mirrored view of the virtual environment is provided via peer-to-peer streaming.

7. The method of claim 1, wherein the first set of data is provided to the second user via asynchronous messaging.

8. The method of claim 1, wherein the selection of at least one of the control tools is received via asynchronous messaging.

9. The method of claim 1, wherein the one or more control tools and the real-time mirrored view are provided via a mobile computing device, the method further comprising:
pairing the mobile computing device and the virtual or augmented reality system.

10. The method of claim 9, wherein pairing comprises authenticating the second user.

11. The method of claim 1, wherein providing the first set of data to the second user comprises:
providing the first set of data to a first trained learning system; and
receiving, from the trained learning system, an adjustment of the one or more parameters.

12. The method of claim 11, further comprising determining, at a second trained learning system, a predicted response of the first user based on the adjustment.

13. The method of claim 12, further comprising determining whether the predicted response passes a predetermined threshold.

14. The method of claim 13, wherein the predetermined threshold is received from an electronic health record database.

15. The method of claim 12, wherein the predicted response corresponds to one or more predicted biometric values from the first user.

16. The method of claim 12, further comprising simulating the predicted response of the first user in response to the adjustment.

17. The method of claim 13, further comprising providing an indication to the second user when the predicted response passes the threshold.

18. A system comprising:
a virtual or augmented reality system, comprising a virtual or augmented reality display adapted to display a virtual environment to a first user;
one or more biometric sensors coupled to the first user;
a computing node comprising a computer readable storage medium having program instructions embodied therewith, the program instructions executable by a processor of the computing node to cause the processor to perform a method comprising:
providing a virtual environment to a first user via a virtual or augmented reality system, wherein the first user is located at a first location;
collecting a first set of data from the one or more biometric sensors based on interaction of the first user with the virtual environment, the first set of data comprising biometric data of the first user as the user engages in a training protocol received from a database;
providing, to a second user via a network, a real-time mirrored view of the virtual environment provided to the first user;
providing the first set of data to the second user via the network;
providing one or more control tools to the second user, the one or more control tools configured to adjust one or more parameters of the virtual environment;
receiving, from the second user, a selection of at least one of the control tools; and
adjusting the virtual environment based on the user selection.

19. A computer program product for providing a virtual or augmented reality platform, the computer program product comprising a computer readable storage medium having program instructions embodied therewith, the program instructions executable by a processor to cause the processor to perform a method comprising:
providing a virtual environment to a first user via a virtual or augmented reality system, wherein the first user is located at a first location;
collecting a first set of data based on interaction with the virtual environment, the first set of data comprising biometric data of the first user as the user engages in a training protocol received from a database;
providing, to a second user via a network, a real-time mirrored view of the virtual environment provided to the first user;
providing the first set of data to the second user via the network;
providing one or more control tools to the second user, the one or more control tools configured to adjust one or more parameters of the virtual environment;
receiving, from the second user, a selection of at least one of the control tools; and
adjusting the virtual environment based on the user selection.
